(19) United States
(12) Patent Application Publication
(10) Pub. No.: US 2006/0241036 A1
(43) Pub. Date: Oct. 26, 2006

(54) Soluble human interleukin 18 receptor-alpha, method of assaying the same, assay kit and medicinal composition

(76) Inventor: Tomoaki Hoshino, Fukuoka (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/566,269

(22) PCT Filed: Jul. 26, 2004
(86) PCT No.: PCT/JP04/10621

§ 371 Date: Jan. 30, 2006

(30) Foreign Application Priority Data
Jul. 30, 2003 (JP) 2003-282375

(51) Int. Cl. A61K 38/17; A61K 48/00; G01N 33/53; C07K 14/715

(52) U.S. Cl. 514/12; 435/7.92; 530/350; 514/44

(57) ABSTRACT

[PROBLEMS] To confirm the presence of a solubilized human IL-18 receptor-α by a novel ELISA method and provide an assay kit and a medicinal composition containing the solubilized human IL-18 receptor-α as the active ingredient. [MEANS FOR SOLVING PROBLEMS] A solubilized human interleukin-18 receptor-α, a method of assaying the solubilized human interleukin-18 receptor-α by an enzyme immunoassay method characterized by using the following antibody (A), a kit for assaying the solubilized human interleukin-18 receptor-α and a medicinal composition containing the solubilized human IL-18 receptor-α. (A) An anti-human interleukin-18 receptor-α monoclonal antibody capable of recognizing the same epitope as an H44 mouse anti-human interleukin-18 receptor-α monoclonal antibody.

### TECHNICAL FIELD

The present invention relates to a soluble human interleukin-18 receptor α that is expected to be used for analysis of the function of human interleukin-18 receptor and used as a drug for treating, for example, interstitial pneumonia, infections, autoimmune diseases such as articular rheumatism (rheumatoid arthritis), and a method for assaying the same, a method for diagnosing autoimmune diseases such as rheumatism, and an assay kit.

### BACKGROUND ART

Interleukin-18 (hereinafter, referred to as “IL-18”) is the cytokine that was discovered in 1995 as an interferon-γ (IFN-γ) inducer, which is produced by macrophages [Nature 378,88-91 (1995)]. The IL-18 is synthesized as a precursor (pro IL-18), and then is cleaved with an interleukin-1β converting enzyme (caspase-1) or the like to be converted to an activated form (mature IL-18). The precursor of a mouse IL-18 is constituted by 192 amino acids and its activated form is constituted by 157 amino acids.

On the other hand, the precursor of a human IL-18 is constituted by 194 amino acids and its activated form is constituted by 158 amino acids.

The IL-18 receptor belongs to the IL-1 receptor family and IL-18Rα and IL-18Rβ are known.

It is known that IL-18 acts on helper T cell type 1 (Th1) or natural killer cell (NK cell) so as to induce the production of IFN-γ, and in addition, that IL-18 enhances the cytotoxic activity by enhancing cytotoxic T cell activity, and thus IL-18 is believed to be an inflammatory cytokine that causes a Th1 response.

Because of these facts, attention has been given to the relations between IL-18 and Crohn disease, multiple sclerosis, and insulin dependent diabetes, which are caused by excessive reaction of Th1.

Furthermore, the present inventor identified that excessive supply of IL-18 causes pulmonary disorders such as interstitial pneumonia and pulmonary fibrosis (WO01/080891).

Therefore, the present inventor hypothesized that controlling interaction between IL-18 and IL-18 receptors would be useful for preventing or treating diseases caused by excessively expressed IL-18, and selected, as a candidate substance, soluble form of IL-18 receptor α, which has not yet been confirmed to be present in vivo, and started developing a method for assaying it.

Conventionally, regarding interleukin-2, the presence of soluble human interleukin-2 receptor a is confirmed, and enzyme-linked immunosorbent assay (ELISA) using two types of monoclonal antibodies that recognize different epitopes at two sites is used as detecting means thereof.

Non-patent document 1: PHARMINGEN OptEIA (TM) Human IL-2sR α (CD25) Set catalog (Catalog #559104)(published on Aug. 17, 2000, PHARMINGEN, San Diego, Calif., USA)

Furthermore, regarding IL-18, it is reported that a combination of artificially produced (recombinant) soluble human IL-18 receptor α and IL-18 receptor P inhibits production of IL-18-induced IFN-γ.

Non-patent document 2: The Combination of Soluble IL-18Rα and IL-18Rβ Chains Inhibits IL-18-Induced IFN-γ (Journal of Interferon and cytokine research 22: P. 593-601, 2002, Mary and Liebert, Inc.)

However, such a soluble receptor is obtained by artificially adding a sequence of signal peptide or the like to an extracellular domain of the IL-18 receptor, assuming that the extracellular domain of the IL-18 receptor is in soluble form, and thus is not a natural soluble receptor. Furthermore, since it is expressed in a mouse cancer cell line, it may be different from those produced by humans in the three-dimensional structure or the carbohydrate structure. That is to say, the presence of natural soluble receptors is not yet known, so that an assay method is also not established.

In addition, it is reported that it is only experiment in vivo and production of IFN-γ can be inhibited only by using a combination of soluble human IL-18 receptor a and , that are created artificially. The report did not suggest that a soluble IL-18 receptor α alone can serve as a therapeutic agent in vivo.

The present inventor tried detecting it with ELISA based on the assumption that the human IL-18 receptor α (a subunit of the human IL-18 receptor) has a soluble form.

Regarding two types of antibodies (capture antibody and detect antibody) to be used, candidates are selected among existing monoclonal antibodies of the human IL-18 receptor α, based on the theory that the human IL-18 receptor α that is not yet soluble and soluble human IL-18 receptor α are similar in their structures. However, it is believed that epitope that serves as a binding site when recognizing the human IL-18 receptor α is common to all of the existing monoclonal antibodies, so that it is impossible to detect it with a so-called sandwich method using two types of the existing monoclonal antibodies.

### DISCLOSURE OF INVENTION

### PROBLEMS TO BE SOLVED BY THE INVENTION

As a result of in-depth research to solve the above-described problems, the present inventor established a method for assaying soluble human IL-18 receptor α, further confirmed that the soluble human IL-18 receptor α is present in vivo, confirmed that the soluble human IL-18 receptor α is useful as a drug for treating diseases and thus achieved the present invention. The object thereof is to select a combination of antibodies which recognize the soluble human IL-18 receptor α and whose epitopes are not common, to provide ELISA and an assay kit using the same, and to provide a medicinal composition having the soluble human IL-18 receptor α as an effective component.

### MEANS FOR SOLVING PROBLEMS

The above-described problems can be solved by the followings: A soluble human IL-18 receptor α; A method for assaying a soluble human IL-18 receptor α with an enzyme-linked immunosorbent assay, wherein an antibody (A) below is used; The method for assaying a soluble human IL-18 receptor α, wherein (A) is (a) below; The method for assaying a soluble human IL-18 receptor α, wherein (a) is either one of (a1) to (a3) below; The method for assaying a soluble human IL-18 receptor α, wherein another antibody is (B) below; The method for assaying a soluble human IL-18 receptor α, wherein (B) is (b) below; The method for assaying a soluble human IL-18 receptor α, wherein a primary antibody in which an antibody (1) below is immobilized and a secondary antibody (2) below are used to detect a soluble human IL-18 receptor α; A method for diagnose autoimmune diseases, wherein any one of the methods for assaying a soluble human IL-18 receptor α is used; A kit for assaying a soluble human IL-18 receptor α, comprising an antibody (A) below as an immobilized antibody or a labeled antibody; A kit for assaying a soluble human IL-18 receptor α, comprising two types of antibodies (1) and (2), one of the antibodies being immobilized and the other being labeled; A medicinal composition comprising at least one selected from the group consisting of (X), (Y) below and genes encoding these as an effective component; A drug for preventing or treating diseases caused by IL-18, rheumatism-related diseases, autoimmune diseases including SLE and infectious diseases, comprising at least one selected from the group consisting of (X), (Y) below and genes encoding these as an effective component; A drug for preventing or treating pulmonary disorders and respiratory diseases, comprising at least one selected from the group consisting of (X), (Y) below and genes encoding these as an effective component; and A medicinal composition comprising (x) or (y) below as an effective component.

(X) soluble human IL-18 receptor α

(Y) protein that is constituted by an amino acid sequence in which one or several amino acids are deleted, substituted or added and has the same activity as the soluble human IL-18 receptor α

(x) human IL-18 receptor α gene

(y) gene that is constituted by a base sequence in which one or several bases are deleted, substituted or added and which codes the protein that has the same activity as the soluble human IL-18 receptor α

(A) anti human IL-18 receptor α monoclonal antibody that can recognize the same epitope as a H44 mouse anti human IL-18 receptor α monoclonal antibody

(a) mouse anti human IL-18 receptor α monoclonal antibody that can recognize the same epitope as a H44 mouse anti human IL-18 receptor α monoclonal antibody

(a1) H44 mouse anti human IL-18 receptor α monoclonal antibody

(a2) MAB840 mouse anti human IL-18 receptor α monoclonal antibody

(a3) 117-10C mouse anti human IL-18 receptor α monoclonal antibody

(B) anti human IL-18 receptor α polyclonal antibody

(b) rabbit anti human IL-18 receptor α polyclonal antibody

(1) anti human IL-18 receptor α monoclonal antibody

(2) anti human IL-18 receptor α polyclonal antibody

In the following, “human IL-18 receptor α” may be referred to as “hIL-18Rα”; “soluble IL-18 receptor α” may be referred to as “sIL-18Rα”; and “soluble human IL-18 receptor α” may be referred to as “shIL-18Rα”;

### EFFECT OF THE INVENTION

The shIL-18Rα of the present invention is expected to be used for analysis of the functions of IL-18 and IL-18R signal, a drug for treating interstitial pneumonia, infections and the like. Furthermore, the method for assaying shIL-18Rα of the present invention is significant in that shIL-18Rα can be assayed, which was conventionally impossible. Moreover, the diagnosis method of the present invention makes it possible to diagnose autoimmune disease such as rheumatics. Furthermore, the kit of the present invention is simple for assay and very useful in a medical field.

### BEST MODE FOR CARRYING OUT THE INVENTION

The shIL-18Rα (X) of the present invention is a protein with a molecular weight of about 60 kDa that is present in human blood serum or the like, and has a binding ability with IL-18 as well as anti hIL-18Rα monoclonal antibody such as H44 mouse anti hIL-18Rα monoclonal antibody, and anti hIL-18Rα polyclonal antibody.

The shIL-18Rα of the present invention can be isolated and purified by the enzyme-linked immunosorbent assay of the present invention as described below or HPLC or affinity column chromatography employing anti IL-18Rα antibody from a living body such as human or mammal blood serum, urine, tissue extracts or the like and bronchoalveolar lavage fluid or the like. Furthermore, a soluble IL-18 receptor can be purified from a culture liquid of a cell line excessively expressing IL-18Rα (e.g., human NK cell NKO cell line or cell line excessively expressing IL-18Rα gene) by HPLC or affinity column chromatography employing anti IL-18Rα antibody.

Furthermore, it can be purified from a supernatant of a stimulated human lymphocyte culture by HPLC or affinity column chromatography employing anti IL-18Rα antibody.

Furthermore, it also can be purified by HPLC or affinity column chromatography employing anti IL-18Rα antibody, after adding protease (e.g., metalloproteinase, TNF-α converting enzyme (TACE)) to a culture liquid of a cell line excessively expressing IL-18Rα (e.g., human NK cell NKO cell line or cell line excessively expressing IL-18Rα gene) or human lymphocyte.

Furthermore, the shIL-18Rα can be synthesized artificially by extraction from gene-recombinant yeast or E. coli or the like, chemical synthesis or the like, based on the analysis of the crystal structure with X-rays or the amino acid sequence of its protein.

As used in the present specification, “the same activity as the shIL-18Rα” of (Y) protein that is constituted by an amino acid sequence in which one or several amino acids are deleted, substituted or added and has the same activity as the shIL-18Rα or (y) gene that is constituted by a base sequence in which one or several bases are deleted, substituted or added and which codes the protein that has the same activity as the shIL-18Rα means the ability of binding to human IL-18 antigen as well as the shIL-18Rα.

In the present invention, examples of (A) anti human IL-18 receptor α monoclonal antibody that can recognize the same epitope as a H44 mouse anti human IL-18 receptor α monoclonal antibody (hereinafter, referred to as “H44 mouse anti hIL-18Rα), which is used as one antibody of enzyme-linked immunosorbent assay, include mouse, rat, rabbit, human or hamster antibodies, but (a) mouse antibody is preferable and more specifically, the following three can be used.

(a1) H44 mouse anti hIL-18Rα

(a2) MAB840 mouse anti human IL-18 receptor α monoclonal antibody(manufactured by R&D Systems, Minneapolis, Minn., USA, hereinafter referred to as MAB840 mouse anti hIL-18Rα)

(a3) 117-10C mouse anti human IL-18 receptor α monoclonal antibody (manufactured by Hayashibara Biochemical Laboratories Inc. Okayama, Japan, hereinafter referred to as 117-10C mouse anti hIL-18Rα)

They can be any antibodies, as long as they can recognize a different epitope from epitope which is recognized by the mouse, rat, rabbit, human or hamster anti hIL-18Rα monoclonal antibody or polyclonal antibody as described below that is used on the other hand.

(a1) H44 mouse anti hIL-18Rα is a known monoclonal antibody and is available from Pharmingen, Serotec, eBioscience.

(a2) MAB840 mouse anti hIL-18Rα is a known monoclonal antibody and is available from R&D Systems (R&D Systems Catalog No. MAB840).

(a3) 117-10C anti hIL-18Rα is a known monoclonal antibody and is available from Hayashibara Biochemical Laboratories Inc. (J. Biol. Chem. Vol. 272, No. 41, Issue of October 10, pp. 25737-25742, 1997)

The anti human IL-18 receptor α polyclonal antibody used in the present invention is a known polyclonal antibody. The polyclonal antibody is a group of antibodies that can recognize different epitopes. The polyclonal antibodies as a whole can recognize various epitopes. The anti human IL-18 receptor α polyclonal antibody used in the present invention can recognize different epitopes from the above-described monoclonal antibody, and it seems that the epitopes recognized are in positions that do not cause steric hindrance with the position of the epitope that is recognized by the anti human IL-18 receptor α monoclonal antibody.

In the assay method of the present invention, other (1) anti human IL-18 receptor α monoclonal antibodies can be used than (A) anti human IL-18 receptor α monoclonal antibody that can recognize the same epitope as a H44 mouse anti human IL-18 receptor α monoclonal antibody (hereinafter, referred to as “H44 mouse anti hIL-18Rα) as described above. In this case, monoclonal antibodies that can recognize different epitopes than the epitope that is recognized by that antibody, (2) anti human IL-18 receptor α polyclonal antibodies or the like can be used.

As (1), mouse antibodies are preferable and as (2), rabbit antibodies are preferable.

The rabbit anti human IL-18 receptor α polyclonal antibodies are available from Dr. Tsukasa Seya (Osaka Medical Center for Cancer and Cardiovascular Diseases, Koizumi H, Sato-Matsumura K C, Nakamura H, Shida K, Kikkawa S, Matsumoto M, Toyoshima K, Seya T. Distribution of IL-18 and IL-18 receptor in human skin; various forms of IL-18 are produced in keratinocytes. Arch Dermatol Res 2001; 293:325-333.)

In the method for assaying shIL-18Rα of the present invention, one kind of the above-described monoclonal antibodies and the above-described polyclonal antibodies can be used, and either can be used as the primary antibody. However, a monoclonal antibody having high selectivity is preferably used in order to be immobilized as a capture antibody. Therefore, also in the kit for assaying shIL-18Rα in vivo sample of the present invention, it is preferable to use either one of the above-described monoclonal antibodies that is immobilized as the primary antibody.

In the present invention, the primary antibody can be immobilized to an insoluble substance such as glass, plastic, particulate, magnetic particulate, and the shape thereof can be a wall of a vessel such as tube, beads, protein particulate, iron particulate, micro plate, filter for immuno chromatography, glass filter and the like, but micro plates are generally used.

In the present invention, antibodies that can be used as the secondary antibody can be detected in the following method, for example.

Biotin is bonded to the secondary antibody, and a sandwich method is performed. Thereafter, streptavidin-bonded horseradish peroxidase is added so that the avidin is bonded to the biotin. An enzyme substrate of horseradish peroxidase is added thereto, and the enzyme activity is measured, so that the soluble human IL-18 receptor α that is captured by the sandwich method is measured.

Besides that, the secondary antibody can be labeled with known radioisotope, enzyme, florescent substance, chemiluminescent substance, coloring substance or the like.

The method for diagnosing autoimmune disease of the present invention can be performed, using the above-described method for assaying soluble human IL-18 receptor α.

Examples of autoimmune diseases include articular rheumatism, interstitial pneumonia, and collagen disease.

For the kit for assaying shIL-18Rα of the present invention, in addition to the primary antibody and the secondary antibody, a target used for immobilization, a label, a buffer and the like can be combined as appropriate. Furthermore, for the kit for assaying shIL-18Rα of the present invention, an acid solution (e.g., 25 mM Glycine with pH3.0, 150 mM NaCl or the like) for separating shIL-18Rα antigen from the antibody can be further added.

The method for assaying shIL-18Rα and the kit for assaying shIL-18Rα of the present invention can be used for assaying shIL-18Rα in vitro, in addition to assaying shIL-18Rα in vivo.

(X) shIL-18Rα or (Y) protein that is constituted by an amino acid sequence in which one or several amino acids are deleted, substituted or added and has the same activity as the shIL-18Rα of the present invention can be used as a drug for preventing or treating diseases caused by IL-18 or a drug for preventing or treating pulmonary disorders, and are useful as various medicinal composition, because they have effects of treating diseases caused by toxin derived from bacteria such as gram-negative and positive bacteria such as gram-negative bacterium E. coli, as described below.

Furthermore, as confirmed in the examples below, there is a large quantity of soluble IL-18 receptors in the blood serum of a TG mouse in which IL-18Rα is excessively expressed in vivo. Therefore, a hIL-18Rα gene is introduced to a human, and hIL-18R is excessively expressed, so that a large amount of shIL-18R can be produced in the blood serum, and thus gene therapy can be achieved with respect to various diseased as described above.

### EXAMPLE 1

(Confirmation of shIL-18Rα Antigen in Blood Serum or BAL by ELISA)

4 μg/ml of H44 mouse anti hIL-18Rα that was dissolved in a PBS buffer was dispensed into ELISA plates (manufactured by Nunc) each in amount of 100 μl/well and left undisturbed over night at 4° C. so that the H44 mouse anti hIL-18Rα, which was the primary antibody, was immobilized to the plates, and then washed with 200 μl of a PBS buffer containing 0.5% Twin 20 (surfactant) twice.

In order to prevent non-specific adhesion of the secondary antibody to the plates, 200 μl/well of Block Ace (manufactured by Dainippon Pharma. Co. Ltd.) were added and left undisturbed for two hours at room temperature for blocking, and then again washed with 200 μl of a PBS buffer containing 0.5% Twin 20 twice. Instead of being left undisturbed for two hours at room temperature, it may be left undisturbed over night at 4° C.

Human blood serum (fourfold dilution) and BAL (undiluted solution of bronchoalveolar lavage fluid), which are samples to be tested, were dispensed to 100 μl/well each. Furthermore as a standard hIL-18Rα antigen solution, 100 μg/ml of recombinant human IL-18Rα (manufactured by R&D) was diluted to prepare diluted solutions by various factors, and each was dispensed to a separate well in a similar manner. After the dispensation, after two hours at room temperature, the standard hIL-18Rα antigen or shIL-18Rα antigen in the blood serum and BAL were bonded to the primary antibody, and then each well was washed with 200 μl of a PBS buffer containing 0.5% Twin 20 three times.

Next, biotin-bonded rabbit anti hIL-18Rα polyclonal antibody solution, which was the secondary antibody, dissolved in 40% Block Ace such that the concentration was 2 μg/ml was dispensed into 100 μl/well, and left undisturbed for 90 minutes at room temperature so that the secondary antibody was bonded to shIL-18Rα antigen, and then each well was washed with 200 μl of a PBS buffer containing 0.5% Twin 20 four times.

0.5 μg/ml of streptavidin-bonded horseradish peroxidase was added to each well in an amount of 100 μl, and left undisturbed for 30 minutes at room temperature so that the avidin was bonded to the biotin, and then each well was washed with 200 μl of a PBS buffer containing 0.5% Twin 20 five times.

100 μl/well of ABTS (ELISA POD Substrate A.B.T.S. kit Nakarai, Kyoto), which is an enzyme substrate of horseradish peroxidase was added and left undisturbed for 30 minutes at room temperature so that an enzyme reaction was caused, and then 100 μL/well of a stop solution was added to stop the enzyme reaction.

The amount of the shIL-18Rα in the blood serum and BAL, which were samples to be tested, were determined by measuring absorbances at 450 nm (main) and 650 nm (sub) and performing comparison with the amount of the standard hIL-18Rα antigen.

The amounts of shIL-18Rα in the blood serum of healthy people were 159.2±89.5 ng/ml (n=45). The amounts of shIL-18Rα in the blood serum of patients with articular rheumatism (RA) were 233.5±109.2 ng/ml (n=34). The amount of shIL-18Rα in the blood serum of patients with articular rheumatism (RA) was significantly higher than that of healthy people (p=0.00216, unpaired Student t-test). The amounts of shIL-18Rα in the blood serum of patients with interstitial pneumonia were 3361.8±2307.3 ng/ml (n=21), and the amounts of shIL-18Rα in BAL were 2.2±1.5 ng/ml (n=33).

These facts indicate that shIL-18Rα is present in the blood serum and BAL. Moreover, the fact that the amount of shIL-18Rα in the blood serum of patients with articular rheumatism or interstitial pneumonia was evidently higher than that of healthy people indicates that the method for assaying shIL-18Rα of the present invention is useful as a method for diagnosing diseases of patients with rheumatism or interstitial pneumonia or other diseases.

(Confirmation of the presence of shIL-18Rα antigen in blood serum and affinity confirmation test)

H44 mouse anti hIL-18Rα antibody was coupled to HiTrap NHS-activated HP column (Pharmacia Biotech Ab, Uppsala, Sweden) and a human blood serum was subjected to affinity column chromatography using this affinity column to confirm the affinity. 10 mM phosphate buffer pH 6.8 was used for the binding buffer, 10 mM phosphate buffer and 50 mM NaCl, pH 6.8 for the washing buffer, 100 mM glycine buffer pH2.5 for elution buffer, and 1 M phosphate buffer pH8.0 for neutralization buffer. A human blood serum sample was diluted to twofold with the binding buffer and filtered with a 0.45 μm filter, and subjected to H44 mouse anti hIL-18Rα antibody affinity column that had been equilibrated with the binding buffer in advance. The affinity column was washed with the washing buffer. The elution buffer was allowed to flow through the column, and every 1 ml of the elution buffer was put into a tube containing 50 μl of neutralization buffer to be collected (fractions collected are denoted by Fraction No. 1, 2, 3, . . . in the order), and the fractions were monitored with UV 280 nm. The collected fractions were dialyzed with a PBS buffer at 4° C. At UV 280 nm, 0.0687, 0.3598, 0.7073, 0.0949, 0.0377, 0, 0 and 0 were Fractions No. 1 to 8, respectively, according to the monitor. The shIL-18Rα in the dialyzed Fractions Nos. 1 to 8 was measured by the above-described sandwich ELISA method, and the results were <200, 1797, 1778, 1259, <200, <200, <200, and <200 ng/ml, respectively. That is, it was confirmed that the shIL-18Rα was present in the Fractions Nos. 2, 3 and 4. Thus the shIL-18Rα that was collected from human blood serum was subjected to H44 mouse anti hIL-18Rα antibody affinity column chromatography to confirm the affinity.

(Antigen Specificity and Molecular Weight Confirmation Test by Western Blotting)

Fractions Nos. 1 to 8 that were dialyzed in the above-described manner were analyzed by western blotting using H44 mouse anti hIL-18Rα antibody. As shown in FIG. 1, in a molecular weight of about 60 kDa in Fractions 2, 3, and 4, the presence of shIL-18Rα that was recognized by the H44 mouse anti hIL-18Rα antibody was confirmed (the presence was confirmed in Fractions Nos. 2 and 4 although they are not distinctive in the drawing). These results match with the results of the above-described affinity column chromatography and sandwich ELISA. The results as described above firmly confirmed the presence of shIL-18Rα in the blood serum.

### EXAMPLE 2

Human lymphocytes were suspended in a cell density of 2×10⁶ cells/mL in RPMI-1640 to which 10% FCS was added. These lymphocytes were not stimulated or stimulated with LPS (100 ng/mL) or with mitogen PMA (10 ng/mL). shIL-18R was detected in 1765 ng/mL, 2793 ng/mL and 2885 ng/mL in the culture supernatant. On the other hand, the amount of shIL-18R in 10% FCS PMI-1640 to which lymphocytes were not added was 170 ng/mL or less. That is, a larger amount of shIL-18R was produced from stimulated lymphocytes. Thus, a soluble IL-18 receptor was obtained from the culture supernatant by stimulating lymphocytes with LPS (100 ng/mL) or with mitogen PMA (10 ng/mL) or the like and performing purification using HPLC or affinity column chromatography with anti IL-18Rα antibody. As a result, it was found out that although shIL-18R was originally present in a cell culture, it was excessively expressed by stimulation with LPS, PMA or the like.

### EXAMPLE 3

[Confirmation of Therapeutic Effect of Soluble IL-18Rα]

(Induction of Excessive Expression of Soluble IL-18Rα in Animal Models)

As shown in FIG. 2, 1.6 Kb of cDNA of a mouse IL-18Rα was inserted in a SnaBI site of a VA-hCD2 vector (Zhumabekov, Journal of Immunological Methods, 185 (1995), 133-140), and cleaved with KpnI/NotI, and the fragment was injected to a fertilized egg of C57BL/6(B6) mouse according to the regular method. Four lines of transgenic (TG) mice No. 17, 27, 51, and 56 were established. Spleen cells were extracted from 10 week old TG mice of these four lines and control wild type (WT) B6 mice (WT#1, WT#2) and were suspended in a cell density of 2×10⁶ cells /mL in RPMI-1640 to which 10% FCS was added.

The samples were cultured in the presence of anti mouse CD3 antibody (1 μg/mL), human IL-2 (200 U/mL), mouse IL-18 (200 ng/mL), human IL-2 (200 U/mL) plus mouse IL-18 (200 ng/mL) for 18 hours, and IFN-γ was measured with an ELISA kit (manufactured by R&D). As shown in FIG. 3, the spleen cells of the TG mice were reacted with the human IL-2 (200 U/mL) plus mouse IL-18 (200 ng/mL) and produced IFN-γ more significantly than WT.

Furthermore, the TG mice expressed IL-18Rα intensely on the lymphocytes more significantly than the WT mice in the membrane surface antigen analysis using anti IL-18Rα antibody.

When soluble IL-18Rα receptor was measured by the ELISA that we established, it was detected in the blood serum of the WT mice in an amount of not more than the detection limit even when measuring by limiting dilution method.

On the other hand, when the blood serum of the TG mice was measured with not less than fourfold dilution by the limiting dilution method, it was possible to detect soluble IL-18Rα receptor. That is, a large amount of soluble IL-18 α receptor was present in the blood serum of the TG mice.

The above-described results proved that in the TG mice that we established,

1. IL-18 receptor α that can react with IL-18 is excessively expressed;

2. a part thereof is converted to be soluble; and

3. a large amount of soluble IL-18 receptor α is present in the blood serum.

In the following experiments, line No. 51 was used. Hereinafter this is referred to as “CD2-IL-18Rα#51”.

(Confirmation of Therapeutic Effect of Excessively Expressed Soluble IL-18Rα Against Toxin)

Ten 8 to 11 week old female TG mice (CD2-IL-18Rα#51) and ten control wild type B6 mice were used. The mice were intraperitoneally administered with lipopolysaccalide (LPS) derived from gram-negative bacterium E. coli (E. coli serotype o55:B5 catalog No. L4005, Sigma [St. Louise, Mo.]) in 40 μg/g weight at day 1. As shown in FIG. 4, the sensitivity to LPS of the TG mice is lower than that of the WT mice. That is, the TG mice were provided with resistance against LPS. This result indicates that excessive soluble IL-18 receptor has a therapeutic effect against toxin (endotoxin, exotoxin) derived from bacteria in vivo.

(Confirmation of Preventive and Therapeutic Effects Against Side Effects of Anticancer Agents by Soluble IL-18Rα)

Five 6 week old female TG mice (CD2-IL-18Rα#51) and five control wild type B6 mice were intraperitoneally administered with 2 mg of BLM (bleomycin) at day 1. Furthermore, the mice were intraperitoneally administered with 2 mg of BLM at day 8. At day 28, 20% formalin was refluxed through the lungs of the mice, which were then fixed. The produced paraffin blocks were subjected to HE blotting. In the TG mice (right in FIG. 5), pulmonary disorders (interstitial pneumonia, pulmonary fibrosis) due to BLM were suppressed more significantly than in the WT mice (left in FIG. 5). This result indicates that excessive soluble IL-18 receptor has a therapeutic effect against pulmonary disorders.

### BRIEF DESCRIPTION OF DRAWINGS

FIG. 1 is a view showing the results of the antigen specificity and the molecular weight confirmation of dialyzed samples by western blotting.

FIG. 2 is a schematic diagram of a gene for producing TG mice containing mouse IL-18Rα gene.

FIG. 3 is a graph showing the amount of IFN-γ produced of spleen cells.

FIG. 4 is a graph showing LPS sensitivities of TG mice and WT mice.

FIG. 5 is a view showing an effect of suppressing pulmonary disorders due to shIL-18Rα.

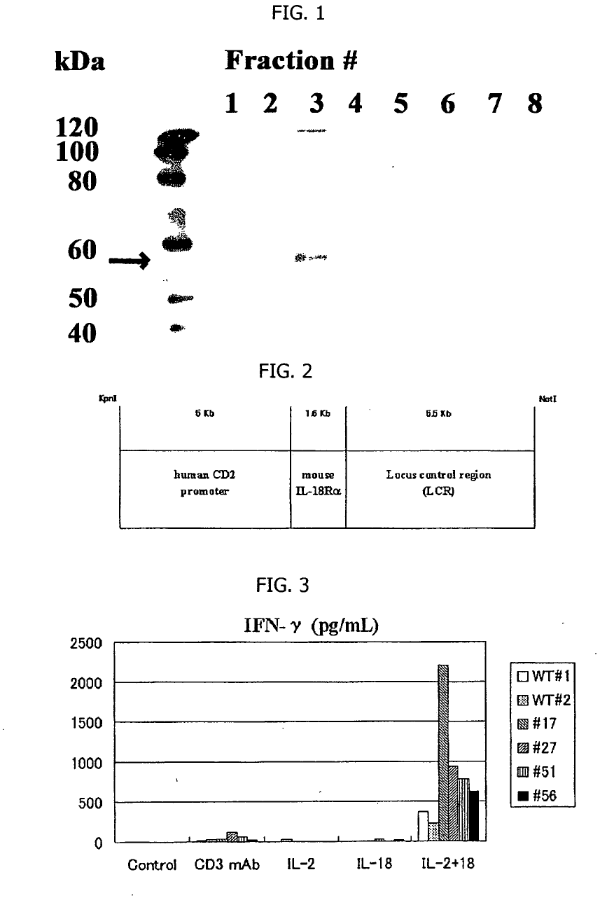

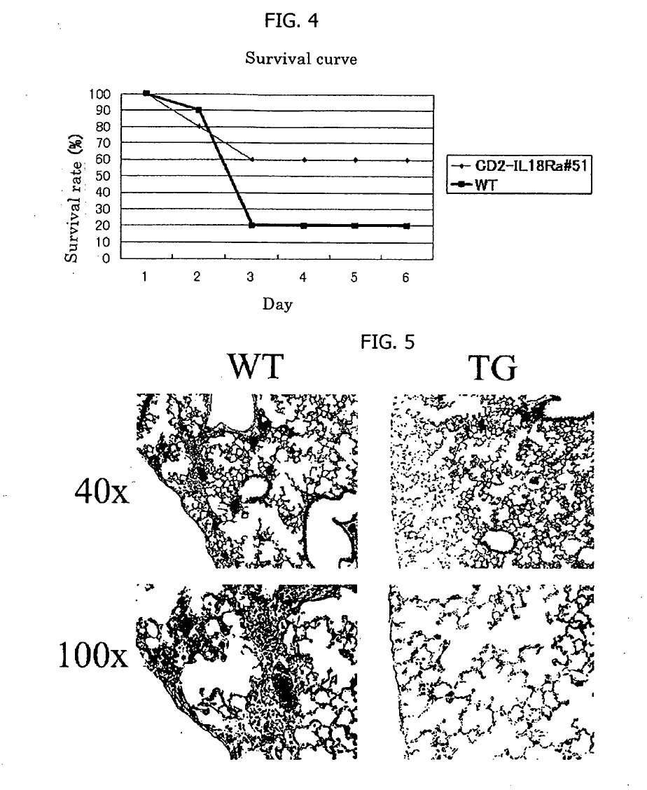

1. A soluble human interleukin-18 receptor α.

2. A method for assaying a soluble human interleukin-1 8 receptor α with an enzyme-linked immunosorbent assay, wherein an antibody (A) is used, wherein
(A) is anti human interleukin-18 receptor α monoclonal antibody that can recognize the same epitope as a H44 mouse anti human interleukin-18 receptor α monoclonal antibody.

3. The method for assaying a soluble human interleukin-18 receptor α according to claim 2, wherein (A) is
(a) mouse anti human interleukin-18 receptor α monoclonal antibody that can recognize the same epitope as a H44 mouse anti human interleukin-18 receptor α monoclonal antibody.

4. The method for assaying a soluble human interleukin-18 receptor α according to claim 3, wherein (a) is either one of (a1) to (a3):
(a1) H44 mouse anti human interleukin-18 receptor α monoclonal antibody,
(a2) MAB840 mouse anti human interleukin-18 receptor α monoclonal antibody, or
(a3) 117-10C mouse anti human interleukin-18 receptor α monoclonal antibody.

5. The method for assaying a soluble human interleukin-18 receptor α according to claim 2, wherein another antibody is
(B) anti human interleukin-18 receptor α polyclonal antibody.

6. The method for assaying a soluble human interleukin-18 receptor α according to claim 5, wherein (B) is
(b) rabbit anti human interleukin-18 receptor α polyclonal antibody.

7. The method for assaying a soluble human interleukin-18 receptor α according to claim 5, wherein a primary antibody in which an antibody (1) below is immobilized and a secondary antibody (2) below are used to detect a soluble human interleukin-18 receptor α, wherein
(1) is anti human interleukin-18 receptor α monoclonal antibody and
(2) is anti human interleukin-18 receptor α polyclonal antibody.

8. A method for diagnose autoimmune diseases, wherein the method for assaying a soluble human interleukin-18 receptor α according to claim 2 is used.

9. A kit for assaying a soluble human interleukin-18 receptor α, comprising an antibody (A) below as an immobilized antibody or a labeled antibody, wherein
(A) is anti human interleukin-18 receptor α monoclonal antibody that can recognize the same epitope as a H44 mouse anti human interleukin-18 receptor α monoclonal antibody.

10. A kit for assaying a soluble human interleukin-18 receptor α, comprising two types of antibodies (1) and (2), one of the antibodies being immobilized and the other being labeled, wherein
(1) is mouse anti human interleukin-18 receptor α monoclonal antibody and
(2) is rabbit anti human interleukin-18 receptor α polyclonal antibody.

11. A medicinal composition comprising at least one selected from the group consisting of (X) and (Y) below and genes encoding these as an effective component, wherein
(X) is soluble human interleukin-18 receptor α, and
(Y) is protein that is constituted by an amino acid sequence in which one or several amino acids are deleted, substituted or added in (X) and has the same activity as the soluble human interleukin-18 receptor α.

12. A drug for preventing or treating diseases caused by interleukin-18, comprising at least one selected from the group consisting of (X) and (Y) below and genes encoding these as an effective component, wherein
(X) is soluble human interleukin-18 receptor α, and
(Y) is protein that is constituted by an amino acid sequence in which one or several amino acids are deleted, substituted or added in (X) and has the same activity as the soluble human interleukin-18 receptor α.

13. A drug for preventing or treating pulmonary disorders, comprising at least one selected from the group consisting of (X) and (Y) below and genes encoding these as an effective component, wherein
(X) is soluble human interleukin-18 receptor α, and
(Y) is protein that is constituted by an amino acid sequence in which one or several amino acids are deleted, substituted or added in (X) and has the same activity as the soluble human interleukin-18 receptor α.

14. A medicinal composition comprising (x) or (y) below as an effective component, wherein
(x) is human interleukin-18 receptor α gene and
(y) is gene that is constituted by a base sequence in which one or several bases are deleted, substituted or added in (X) and which codes the protein that has the same activity as the soluble human interleukin-18 receptor α.
